(19) United States
(12) Patent Application Publication
(10) Pub. No.: US 2015/0216825 A1
(43) Pub. Date: Aug. 6, 2015

(54) ORGANIC ACIDS AS GROWTH INHIBITORS OF PATHOLOGICAL CALCIFICATION AND USES THEREOF

(71) Applicants: Jeffrey D. Rimer, Houston, TX (US); John Asplin, Chicago, IL (US)

(72) Inventors: Jeffrey D. Rimer, Houston, TX (US); John Asplin, Chicago, IL (US)

(73) Assignee: University of Houston System, Houston, TX (US)

(21) Appl. No.: 14/616,035

(22) Filed: Feb. 6, 2015

Related U.S. Application Data
(60) Provisional application No. 61/936,542, filed on Feb. 6, 2014.

(51) Int. Cl. A61K 31/194

(52) U.S. Cl. CPC A61K 31/194

(57) ABSTRACT

In an embodiment of the present disclosure, there is provided a composition for inhibiting calcium oxalate crystal growth. In an embodiment of the present disclosure, there is provided a composition for inhibiting calcium phosphate crystal growth. In some embodiments, such a composition comprises at least one citrate derivative of an organic acid and at least one pharmaceutically acceptable carrier. In another embodiment, the present disclosure relates to a method of controlling calcium oxalate crystal growth in a subject in need thereof. Such a method comprises administering to the subject an effective amount of the aforementioned composition. In another embodiment, the present disclosure pertains to a method of treating kidney stone disorder. Such a method comprises administering to a subject in need thereof a therapeutically effective amount of the aforementioned composition. In yet another embodiment, the present disclosure relates to a method of treating calcium oxalate stone disease. In an embodiment, the method comprises administering to a subject in need thereof a therapeutically effective amount of the aforementioned composition.

### CROSS REFERENCE TO RELATED APPLICATION

This application claims priority to U.S. Provisional Application No. 61936,542 file on Feb. 6, 2014. The entirety of the aforementioned application is incorporated herein by reference.

### STATEMENT REGARDING FEDERALLY SPONSORED RESEARCH

This invention was made with government support under the National Science Foundation Award (Grant No. 1207441). The government has certain rights in the invention.

### BACKGROUND

Calcium oxalate is the most common constituent of urinary calculi and relatively large crystals of this salt are frequently found in freshly voided urine from patients with recurrent calcium-containing stones. Current treatments of calcium oxalate stone disease include water intake, diet supervision, and alkalization agents, which collectively reduce calcium oxalate super saturation in urine. Hydrochlorothiazide, sodium potassium phosphate, potassium citrate, and allopurinol are drugs available for the treatment of calcium oxalate stone disease and reported to reduce its recurrence. While these treatments can be effective, they do not completely prevent stone recurrence. In addition, many of the current treatments have significant adverse effects. Therefore, there is a need to develop more effective drugs for preventing calcium oxalate stone formation with fewer side effects.

### BRIEF SUMMARY

In some embodiments, the present disclosure provides a composition for inhibiting calcium oxalate crystal growth. In an embodiment, the composition inhibits growth of crystals comprising calcium oxalate monohydrate. In another embodiment, the composition inhibits growth of crystals comprising calcium oxalate dihydrate. In yet another embodiment, the composition inhibits growth of crystals comprising calcium oxalate trihydrate. In another embodiment, the composition inhibits growth of crystals comprising calcium oxalate monohydrate and calcium oxalate trihydrate. In an embodiment, such a composition comprises at least one citrate derivative of an organic acid and at least one pharmaceutically acceptable carrier. In some embodiments the citrate derivative of an organic acid is hydroxycitrate. In an embodiment the citrate derivative of an organic acid is tricarballylic acid. In another embodiment the citrate derivative of an organic acid is isocitrate. In an embodiment of the present disclosure the composition further comprises a urinary protein. In some embodiments the composition for inhibiting growth of crystals comprising calcium oxalate comprises two citrate derivatives of an organic acid and at least one pharmaceutically acceptable carrier. In a preferred embodiment, the two citrate derivatives of an organic acid are hydroxycitrate and tricarballylic acid. In another embodiment, the two citrate derivatives of an organic acid are hydroxycitrate and isocitrate. In a preferred embodiment, the composition is in a form suitable for oral administration.

In another embodiment, the present disclosure provides a composition for inhibiting calcium phosphate crystal growth.

In another embodiment of the present disclosure, there is provided a method of controlling calcium oxalate crystal growth in a subject in need thereof. In an embodiment, the method is for controlling growth of crystals comprising calcium oxalate monohydrate. In another embodiment, the method is for controlling growth of crystals comprising calcium oxalate dihydrate. In yet another embodiment, the method is for controlling growth of crystals comprising calcium oxalate trihydrate. In yet, still another embodiment, the method is for controlling growth of crystals comprising calcium oxalate monohydrate and calcium oxalate dihydrate. In an embodiment, the method comprising administering to the subject an effective amount of a composition comprising at least one citrate derivative of an organic acid and at least one pharmaceutically acceptable carrier. In some embodiments the citrate derivative of an organic acid is hydroxycitrate. In an embodiment the citrate derivative of an organic acid is tricarballylic acid. In another embodiment the citrate derivative of an organic acid is isocitrate. In some embodiments the composition for inhibiting growth of crystals comprising calcium oxalate comprises two citrate derivatives of an organic acid and at least one pharmaceutically acceptable carrier. In an embodiment of the present disclosure the composition further comprises a urinary protein. In some embodiments, the subject has kidney stone disorder. In another embodiment, the subject has renal calcification disorder. In yet another embodiment, the subject has biomineralization induced disease. In a related embodiment, the method comprises administering the aforementioned composition post lithotripsy. In an embodiment, the administration of the aforementioned composition post lithotripsy prevents growth of any remaining crystal fragments.

In some embodiment of the present disclosure, there is provided a method of treating kidney stone disorder. Such a method comprises administering to a subject in need thereof a therapeutically effective amount of the aforementioned composition.

In an embodiment, the present disclosure relates to a method of treating calcium oxalate stone disease. In some embodiment, such a method comprises administering to a subject in need thereof a therapeutically effective amount of the aforementioned composition.

As set forth in more detail herein, the methods and compositions of the present disclosure provide numerous improvements in methods of treating, preventing, or inhibiting calcium oxalate and calcium phosphate crystal growth and related stone diseases.

### BRIEF DESCRIPTION OF THE FIGURES

FIG. 1 shows the characteristic habit of calcium oxalate monohydrate (COM) crystals with (100) basal surfaces, (010) side faces, and apical tips expressed by {12-1} and {021} faces. Crystal indexing is based on the P2₁/c space group with cell parameters a=6.290 A°, b=14.580 A°, and c=10.116 A° (β=109.46A°).

FIG. 2 shows structural mapping of the organic growth modifiers (OGMs) that were used in this study, which include: 1-oxalic acid (OA), 2-malonic acid (MA), 3-succinic acid (SA), 4-glutaric acid (GA), 5-adipic acid (AA), 6-malic acid (MAL), 7-tartaric acid (TTA), 8-tricarballylic acid (TCA), 9-hydroxy methylglutaric acid (HMGA), 10-citric acid (CA), 11-isocitric acid or isocitrate (isoCA), 12-hydroxycitric acid or hydroxycitrate (HCA), 13-dimethyl hydroxyglutaric acid (DHG), 14-butanetetracarboxylic acid (BTCA).

FIG. 3 shows COM crystal morphology characterized by (top) optical microscopy and (bottom) scanning electron microscopy, or SEM. (Left) Images of the control crystals in the absence of any growth inhibitor. These crystals exhibit the characteristic hexagonal platelet bulk morphology illustrated in FIG. 1. (Right) In the presence of 60 μg/mL hydroxycitrate (HCA), the binding of the growth inhibitor to the apical tips results in the formation of COM crystals with diamond-shaped morphology. All optical microscope images were observed in reflectance mode and the SEM images were coated with 15 nm of Pt/Pd;

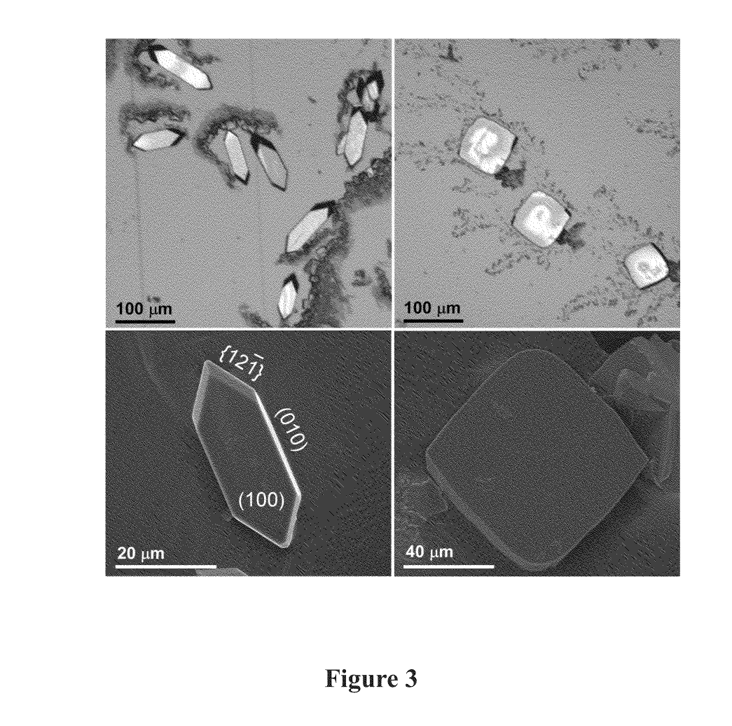

FIGS. 4A-4L show optical micrographs of COM crystals grown in the presence of 60 μg/mL of the following additives: (FIG. 4A) MA, (FIG. 4B) SA, (FIG. 4C) GA, (FIG. 4D) AA, (FIG. 4E) MAL, (FIG. 4F) TTA, (FIG. 4G) DHG, (FIG. 4H) BTCA, (FIG. 4I) TCA, (FIG. 4J) CA, (FIG. 4K) isoCA, and (FIG. 4L) HCA. The chemical names of each acronym are provided in FIG. 2. In some cases the organic crystal growth modifier appeared to reduce the number of nuclei, which resulted in a population of fewer, albeit slightly larger crystals. All images were taken under reflectance mode. Scale bars equal 50 μm;

FIG. 5 shows the length-to-width aspect ratio of COM crystals is measured from microscopy data as the ratio of dimensions along the [001] and [010] directions, respectively, as illustrated in the SEM image (top). The aspect ratio of COM crystals prepared in the absence of OGMs (control) is compared to those prepared in the presence of 60 μg/mL of each OGM. Each acronym is defined in FIG. 2. Error bars equal one standard deviation;

FIGS. 6A-6F shows scanning electron micrographs of COM crystals grown in the presence of different concentrations of citrate (CA) and hydroxycitrate (HCA). In the presence of 20 μg/mL CA, the crystals exhibited little change in morphology compared to that of control (FIG. 6A). In the presence of 60 μg/mL CA, one of apical tips started to become rounded (FIG. 6B). In the presence of 100 μg/mL CA, both apical tips became rounded (FIG. 6C). In the presence of 20 μg/mL HCA, the aspect ratio of crystals was reduced (FIG. 6D). In the presence of 60 μg/mL HCA, crystals revealed diamond-shaped morphology indicating strong specific binding of HCA to the apical tips (FIG. 6E). A higher concentration of HCA (100 μg/mL) did not lead to appreciable changes in the overall morphology compared to studies at 60 μg/mL, although a population of crystals appeared less defined in habit (as illustrated in this panel) (FIG. 6F). In addition, the number of crystals significantly decreased with increasing HCA concentration, which suggests that HCA inhibits COM nucleation. Scale bars in all SEM images equal 50 μm;

FIGS. 7A-7B show kinetic studies of COM crystallization. Kinetic studies were performed using a calcium ion selective electrode (ISE) to measure the depletion of free Ca²⁺ concentration. Raw data acquired from an ISE measurement shows temporal depletion of calcium ion concentration (ppm) during COM crystallization (FIG. 7A). Difference between the concentration at the initial point and the concentration at each time point (using the date in panel A) was used to estimate the COM growth rate and compare the effectiveness of growth inhibitors (FIG. 7B). This procedure was repeated for each OGM multiple times to obtain an average rate of depletion, which is estimated from the initial slope (i.e. within the first 30 min);

FIG. 8 shows the percent inhibition of COM crystallization is measured as {1−r(OGM)/r(control)}×100% where r is the rate of calcium depletion measured by ISE studies (as demonstrated in FIG. 7). The addition of a growth inhibitor results in a decreased rate of COM crystal growth. Here the effects of OGMs at a concentration of 60 μg/mL OGM were compared. Each experiment was measured in situ using supersaturated calcium oxalate solutions at 25° C. Data were averaged from at least five different measurements. Error bars equal one standard deviation. The results reveal that HCA exhibits nearly twice the percent inhibition as CA. The inhibitors TCA and isoCA exhibit nearly equal efficacy as CA (i.e. ˜30%). The inhibitors TTA, DHG, and BTCA are less effective than CA, but were capable of producing a percent inhibition between 10 to 20%;

FIG. 9 shows percent inhibition of selected OGMs (dicarboxylic acids) compared in terms of the number of carbons in their backbone (from C₃ to C₆). Error bars equal one standard deviation. These results suggest the OGMs tested here are not effective inhibitors of COM crystallization, nor is there any apparent pattern in the percent inhibition with increasing carbon number;

FIGS. 10A-10B show percent inhibition of selected OGMs was compared in terms of the number of hydroxyl groups in their structures. Comparison of OGMs with a C₄ backbone (SA, MAL, and TTA) revealed that the insertion of one hydroxyl group reduced the efficacy whereas two hydroxyl groups improved the efficacy (FIG. 10A). Comparison of OGMs with a C₅ backbone revealed that the resulting change in percent inhibition from the insertion of a hydroxyl group very much depends on its location (FIG. 10B). Data are the average of five or more ISE measurements. Error bars equal one standard deviation;

FIGS. 11A-11B show percent inhibition of selected OGMs was compared in terms of the number of carboxylic acid groups in their structures. Comparison of OGMs with a C5 backbone (GA and TCA) revealed that the insertion of one carboxylic acid group significantly increased the efficacy (FIG. 11A). Comparison of OGMs with a C6 backbone (AA and BTCA) revealed that the insertion of two carboxylic acid groups had less effect on crystal growth than the insertion of one carboxylic acid group of the C5 modifiers (FIG. 11B). Data are averages of five or more ISE measurements. Error bar equals one standard deviation;

FIG. 12 shows percent inhibition of COM crystallization in the presence of four OGMs (TCA, isoCA, CA and HCA). The molecule structures shown on the right reveal that each OGM contains three carboxyl groups with varying degree of alcohol substitution. The effect of HCA is significantly larger than the other three modifiers (despite their similar molecular structures). This illustrates the subtle nuance of tailoring OGM efficacy via the insertion (or removal) of a single functional group. Error bar equals one standard deviation;

FIGS. 13A-13B show in situ atomic force microscopy (AFM) deflection mode images of growth hillock on the (010) face of COM crystals. The rectangular hillocks are bounded by {12-1} and {021} steps. A screw dislocation propagates in both the [12-1] and [021] directions in the absence of growth inhibitor (control experiment) (FIG. 13A). In the presence of 0.1 μg/mL HCA, the rate of step advancement in both the [12-1] and [021] directions showed ca. 140% decrease (FIG. 13B). The scale bar equals to 1 μm for both images;

FIG. 14 shows the effect of hydroxycitrate on the upper limit of metastability of a human urine sample. The red line shows the change in absorbance over time, the inflection point is caused by an increase in turbidity which represents nucleation of calcium oxalate crystals in urine. The green and purple lines are assays run with the same human urine containing hydroxycitrate (1 Mm). The addition of hydroxycitrate significantly delays the increase in turbidity, representing an increase in the upper limit of the metastability. Error bar equals one standard deviation.

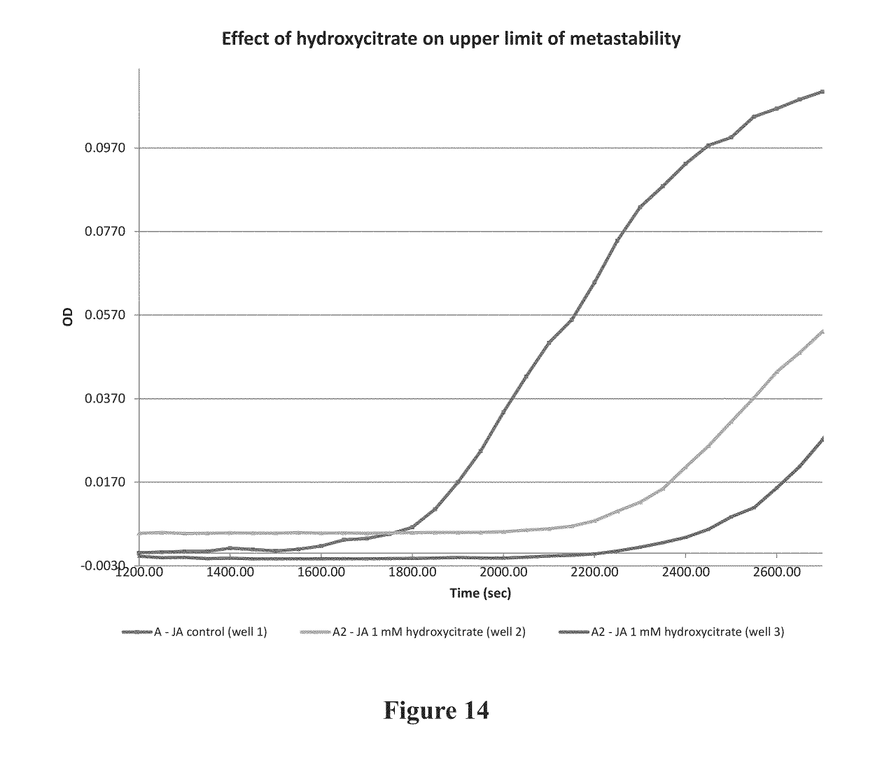

FIG. 15 shows the delay in nucleation (an increase in the upper limit of metastability) for four organic anions: citrate (CT), hydroxycitrate (HCA), isocitrate (ICA), and tricarballylate (TCA), all at a concentration of 1 mM. The results represent a mean of four human urine samples assayed. Hydroxycitrate demonstrates the greatest crystallization inhibition.

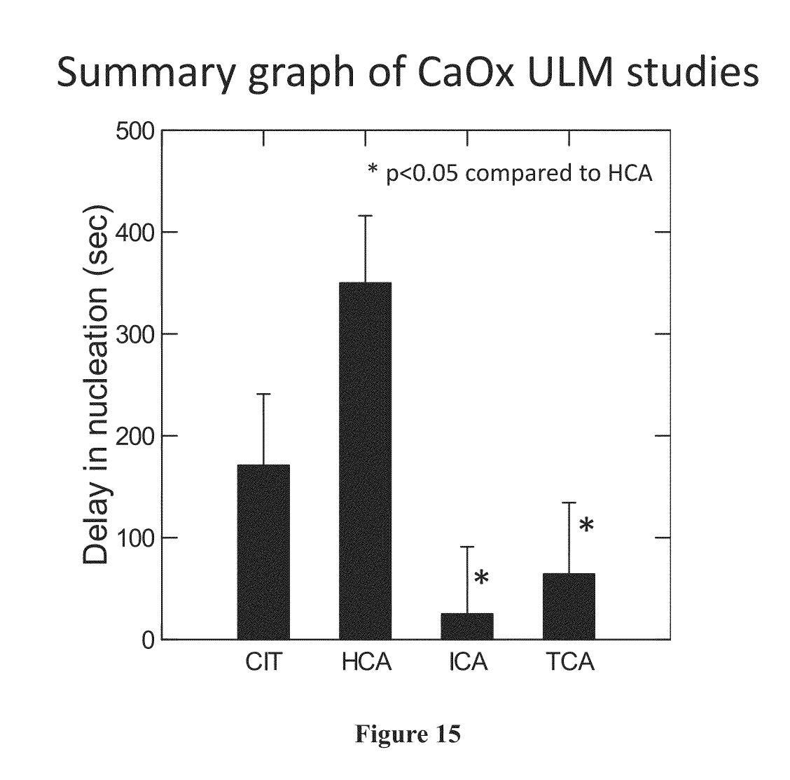

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The following definitions are provided for specific terms which are used in the following written description.

Unless defined otherwise, all technical and scientific terms used herein have the meaning commonly understood by a person skilled in the art to which the invention belongs. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} ed., R. Reigers et al. (eds.), Springer Verlag (1991); and Hale & Marham, The Harper Collins Dictionary of Biology (1991).

The term “treating” or “treatment” as used herein is meant to refer to the administration of a compound or composition according to the present invention to prevent the formation of new kidney stones or to slow the growth of stones that are already present in the kidneys.

Reference herein to “therapeutic” and “prophylactic” is to be considered in their broadest contexts. The term “therapeutic” does not necessarily imply that a mammal is treated until total recovery. Similarly, “prophylactic” does not necessarily mean that the subject will not eventually contract a disease condition. Accordingly, therapy and prophylaxis include amelioration of the symptoms of a particular condition or preventing or otherwise reducing the risk of developing a particular condition. The term “prophylaxis” may be considered as reducing the severity of onset of a particular condition. Therapy may also reduce the severity of an existing condition or the frequency of acute attacks.

As used herein, the term “Subject” includes animals and humans requiring intervention or manipulation due to a disease state, treatment regimen or experimental design.

The present disclosure pertains to organic molecules that are effective inhibitors of calcium oxalate crystallization, which is the most common constituent of human kidney stones (nephrolithiasis). Specifically, the present disclosure pertains to the use of hydroxycitrate and its synthetic analogues as growth inhibitors of calcium oxalate and calcium phosphate (CaP) crystallization, which is another common kidney stone constituent. Current therapies for stone disease include citrate supplements. Citrate is a small organic molecule that is a moderate inhibitor of calcium oxalate crystallization and it complexes with calcium in solution. However, the citrate has some undesirable effects. Upon ingestion, almost all citrate is metabolized to bicarbonate resulting in systemic alkalinization. The alkali load reduces renal tubular reabsorption of filtered citrate resulting in increased excretion of citrate in the urine. The alkali load also increases the pH of urine, which can have undesirable side effects (e.g. higher pH promotes the formation of calcium phosphate). In contrast, the calcium oxalate crystallization inhibitors disclosed herein circumvent the negative side effects of citrate while at the same time provide a more effective inhibition of calcium oxalate crystallization.

Crystallization is ubiquitous in biological systems where interactions between inorganic (salt, ions, etc.) and organic components (proteins, lipids, etc.) often mediate physiological processes in the human body, such as bone and teeth formation. Under abnormal physiological conditions, mineralization can lead to such pathologies as atherosclerotic plaques or vascular calcifications, kidney or gallstones, gout, and osteoarthritis. Small molecules that inhibit abnormal biomineralization are potentially effective therapies against such conditions.

Kidney stone disease is a common pathological disorder. Approximately 10-15% of the U.S. population will have a kidney stone during their lifetime, with incidence rates that are on the rise. Kidney stone pathogenesis is a complex process that involves a series of steps operating either singularly or synergistically to produce polycrystalline aggregates in the kidney. Supersaturated calcium oxalate in urine facilitates crystal nucleation and growth. The growth and aggregation of crystals and the cumulative retention of crystals and/or aggregates leads to the formation of a crystalline mass that is of sufficient size to have clinical significance, i.e., is a kidney stone. Inhibiting one or more of the critical pathways of calcium oxalate stone pathogenesis, including nucleation, growth, aggregation, and retention, via the addition of external agents may potentially serve as an effective therapy for this disease.

It is well established that certain urinary constituents, such as proteins act as natural inhibitors of calcium oxalate crystallization. A common binder group of calcium oxalate crystal inhibitors (i.e. urinary proteins and their synthetic analogues) is carboxylic acid, which binds to oxalate vacancies on crystal surfaces via calcium bridges, _{(COM)}COO⁻ . . . Ca²⁺ . . . ⁻OOC_{(inhibitor)}. Several mechanisms of crystal growth inhibition have been proposed. Crystal growth inhibition may occur through the adsorption of small molecules to surfaces of crystals growing by classical nucleation and spreading of layers (so called layer-by-layer growth). Inhibitors that bind to different sites on a crystal surface (i.e., kinks, ledges, and terraces) reduce step advancement normal to that surface. Inhibitors can therefore serve to retard crystal growth, with implications in therapies for biomineralization-based diseases, or alter growth rates of specific faces, with implications in crystal shape engineering for design of advanced materials. In an embodiment, the calcium oxalate crystallization inhibitors, disclosed herein, bind preferentially to both the {12-1} and {021} surfaces of the calcium oxalate crystals, which leads to a morphological change from thin hexagonal slabs (in the absence of the inhibitor) to thinner diamond slabs.

A current therapy for COM kidney stone disease is citrate, which complexes calcium and is a moderately effective inhibitor of COM crystallization. Applicants' have conducted in vitro studies that reveal that the efficacy of citrate is nearly one-half that of HCA. Moreover, citrate is metabolized upon ingestion, whereas HCA exhibits higher bioavailability. Another advantage of using HCA is that it is FDA approved for use as a component of dietary supplements and therefore is non-toxic. In addition, organic acids used in combination therapies may be an even more effective approach.

In some embodiments, the present disclosure pertains to calcium oxalate crystallization inhibitors. In some embodiments of the present disclosure the calcium oxalate crystallization inhibitor is a citrate derivative of an organic acid. In some embodiments of the present disclosure, citrate derivative of an organic acid may be a citrate-based molecular analogue. In an embodiment of the present disclosure, the citrate-based molecular analogue is hydroxycitrate (HCA). In another embodiment of the present disclosure, the citrate analogue may be isocitrate or tricarballylic acid. In some embodiments the composition for inhibiting growth of crystals comprising calcium oxalate comprises two citrate derivatives of an organic acid and at least one pharmaceutically acceptable carrier. In a preferred embodiment, the two citrate derivatives of an organic acid are hydroxycitrate and tricarballylic acid. In another embodiment, the two citrate derivatives of an organic acid are hydroxycitrate and isocitrate. In another embodiment of the present disclosure, the calcium oxalate crystallization inhibitor is a composition comprising of at least one citrate derivative of an organic acid and a urinary protein. In an embodiment, the composition inhibits growth of crystals comprising calcium oxalate monohydrate. In another embodiment, the composition inhibits growth of crystals comprising calcium oxalate dihydrate. In yet another embodiment, the composition inhibits growth of crystals comprising calcium oxalate trihydrate. In another embodiment, the composition inhibits growth of crystals comprising calcium oxalate monohydrate and calcium oxalate dihydrate. In an embodiment, the calcium oxalate crystallization inhibitor is a composition comprising citrate and a citrate-based molecular analogue. In all embodiment, the molecules disclosed herein, inhibit crystal growth rates by as much as 60%.

The present disclosure relates to the use of HCA and additional organic acids (e.g. isocitrate and tricarballylic acid) as potential drugs for kidney stone disease. The present disclosure also relates to combinations therapy as a potentially more effective approach based on the use of two or more of inhibitor molecules, including citrate and other organic acids (e.g. citrate-based molecular analogues, such as HCA) or citrate and urinary proteins.

In an exemplary embodiment, the calcium oxalate inhibitors disclosed herein may be used to treat the great majority of patients suffering from recurrent stone disease, since over 75% of all renal stones contain calcium oxalate. For example, the calcium oxalate inhibitors may be useful to prevent formation of stones by preventing or inhibiting nucleation, growth and/or aggregation of crystals. The calcium oxalate inhibitors disclosed herein may also be used to treat these patients following extracorporeal shockwave lithotripsy to help ensure passage in the urine of shattered stone particles and renal crystal deposits. The calcium oxalate inhibitors of the present disclosure may also be effective in treating patients with primary hyperoxaluria (a genetic disease resulting in massive over-production of oxalic acid) many of whom suffer total loss of renal function in the early years of life. This could result in less frequent use of the lithotripter and other techniques now used to remove kidney stones. The calcium oxalate inhibitors of the present disclosure may also have use in treating patients post renal transplantation in order to prevent calcium oxalate deposition in the renal graft since many of these patients have substantial body stores of calcium oxalate following long-term dialysis. Finally, it is also contemplated that the inhibitors disclosed herein may be used to treat patients suffering from systemic oxalosis, i.e., deposition of calcium oxalate crystals in many tissues of the body. The stones being treated (or the formation of which is to be prevented) may be present in the kidney, bladder and/or urinary tract. In a further embodiment, it is also possible that other forms of treatment can be used in conjunction with administration of the compound of the present invention, i.e., lithotripsy treatment to break up the stones can be utilized on a patient who has been treated with the calcium oxalate inhibitors disclosed herein.

The calcium oxalate crystal growth inhibitors disclosed herein may be used for the treatment and/or prevention of calcium oxalate stone disease by administering therapeutically effective amounts of the inhibitor to a subject in need thereof.

Accordingly, one aspect of the present disclosure that will be disclosed in more detail herein provides a composition for inhibiting calcium oxalate crystal growth. In an embodiment, the calcium oxalate crystal comprises of calcium oxalate monohydrate. In another embodiment, the calcium oxalate crystal comprises of calcium oxalate dihydrate. In yet another embodiment, the calcium oxalate crystal comprises of calcium oxalate trihydrate. In some embodiments, the calcium oxalate crystal comprises of calcium oxalate monohydrate and calcium oxalate trihydrate. Such a composition comprises at least one citrate derivative of an organic acid and at least one pharmaceutically acceptable carrier. In an embodiment of the present disclosure, citrate derivative of an organic acid is hydroxycitrate. In another embodiment, the citrate derivative of an organic acid is tricarballylic acid. In yet, another embodiment of the present disclosure, the citrate derivative of an organic acid is isocitrate. In some embodiments of the present disclosure the composition comprises at least two citrate derivatives of an organic acid. In an embodiment of the present disclosure, the at least two citrate derivatives of an organic acid are hydroxycitrate and tricarballylic acid. In another embodiment of the present disclosure, the at least two citrate derivatives of an organic acid are hydroxycitrate and isocitrate. In an embodiment of the present disclosure, the composition comprises at least one citrate derivative and a urinary protein. In an embodiment of the present disclosure the composition is in a form suitable for oral administration.

In another embodiment, the present disclosure provides a method of controlling calcium oxalate crystal growth in a subject in need thereof comprising administering to the subject an effective amount of the aforementioned composition. In yet another embodiment, the calcium oxalate crystal is a calcium oxalate trihydrate. In an embodiment of the present disclosure, the subject may have kidney stone disorder. In another embodiment of the present disclosure, the subject may have renal calcification disorder. In yet, another embodiment, the subject may have a biomineralization induced disease. In an embodiment, the method further comprises administering the aforementioned composition post lithotripsy. In an embodiment, the administration of the aforementioned composition post lithotripsy prevents growth of any remaining crystal fragments.

In an embodiment, the present disclosure provided for a method of treating kidney stone disorder comprising administering to a subject in need thereof a therapeutically effective amount of the aforementioned composition.

In some embodiments, the present disclosure relates to a method of treating calcium oxalate stone disease comprising administering to a subject in need thereof a therapeutically effective amount of the aforementioned composition. In an additional embodiment, the method further comprises administering to the subject one or more additional therapeutic agents. Specifically, the one or more additional therapeutic agents are selected from the group consisting of 1-oxalic acid, 2-malonic acid, 3-succinic acid, 4-glutaric acid, 5-adipic acid, 6-malic acid, 7-tartaric acid, 8-tricarballylic acid, 9-hydroxy methylglutaric acid, 10-citric acid (or citrate), 11-isocitric acid (or isocitrate), 12-hydroxycitric acid (or hydroxycitrate), 13-dimethyl hydroxyglutaric acid, 14-butanetetracarboxylic acid (BTCA).

### Applications and Advantages

Calcium oxalate is the most common type of human kidney stone, being the major component of approximately 75% of stones. The approximate number of people affected in the U.S. alone is calculated to be 10 million. In the past 30 years, there has been no major advancement in this area of research; however there are predictions that stone disease is on the rise, and thus the discovery of new and improved drugs that inhibit calcium oxalate crystallization will be paramount. The present disclosure relates to organic acids that are citrate derivatives, as calcium oxalate crystallization inhibitors. Applicants have identified two molecules with equal or slightly less efficacy than citrate (i.e. tricarballylic acid and isocitrate, respectively). In addition, Applicants have found one molecule that is much more effective than citrate in inhibiting calcium oxalate monohydrate (COM) crystallization, which is hydroxycitrate. In vitro studies reveal that hydroxycitrate exhibits nearly twice the percent inhibition of COM crystallization than citrate. Moreover, hydroxycitrate is currently available over the counter as a component of dietary supplements and therefore has an established nontoxicity. Additional benefits of hydroxycitrate is the fact that this molecule is not metabolized (or at least not to the same extent as citrate) and it does not result in substantial increases in urine pH. The latter is of interest for CaP stone formation. For instance, a drug that can also inhibit calcium oxalate and/or calcium phosphate crystal formation without increasing urine pH would be preferential for treatment of CaP stones. The present disclosure also pertains to the use of organic acids in combination as a therapy for renal stone disease.

The preferred composition depends on the method of administration, and typically comprises one or more conventional pharmaceutically acceptable carriers, adjuvants, and/or vehicles (together referred to as “excipients”). Formulation of drugs is generally discussed in, for example, Hoover, J., Remington's Pharmaceutical Sciences (Mack Publishing Co., 1975) and Ansel's Pharmaceutical Dosage Forms and Drug Delivery Systems (Lippincott Williams & Wilkins, 2005).

Solid dosage forms for oral administration include, for example, capsules, tablets, pills, powders, and granules. In such solid dosage forms, the compounds or salts are ordinarily combined with one or more excipients. If administered orally, the compounds or salts can be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in, for example, a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also can comprise, for example, wetting, emulsifying, suspending, flavoring (e.g., sweetening), and/or perfuming agents.

It will be understood by those skilled in the art that the compounds of the invention may be administered in the form of a composition or formulation comprising pharmaceutically acceptable carriers and/or excipients.

Formulations for parenteral administration may, for example, be prepared from sterile powders or granules having one or more of the excipients mentioned for use in the formulations for oral administration. A compound or salt of the invention can be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

Pharmaceutically acceptable carriers are materials useful for the purpose of administering the extract, which are preferably non-toxic, i.e., safe for human intake, and may be solid, liquid or gaseous material, which are otherwise inert and medically acceptable and are compatible with the active ingredient. The pharmaceutically acceptable carrier may be a carrier of a type which has been or would be approved by the Food and Drug Administration for administration to human subjects. The pharmaceutical composition may also contain other active ingredients. For oral administration, fine powders or granules may contain diluting, dispersing and/or surface active agents, and may be presented in a draught, in water or in a syrup; in capsules in the dry state or in a non-aqueous solution or suspension, wherein suspending agents may be included; in tablets, wherein binders and lubricants may be included; or in a solution or suspension in water or other liquid or a syrup. Where desirable or necessary, flavoring, preserving, suspending, thickening or emulsifying agents may be included. Tablets and granules are preferred and these may be coated. Other excipients and modes of administration known in the pharmaceutical art also may be used.

Routes of administration include, but are not limited to oral, dermal, inhalation, injection and intravenous.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved and (b) the limitations inherent in the art of compounding.

Procedures for the preparation of dosage unit forms and topical preparations are readily available to those skilled in the art from texts such as Pharmaceutical Handbook. A Martindale Companion Volume Ed. Ainley Wade Nineteenth Edition The Pharmaceutical Press London, CRC Handbook of Chemistry and Physics Ed. Robert C. Weast Ph. D. CRC Press Inc.; Goodman and Gilman's; The Pharmacological basis of Therapeutics. Ninth Ed. McGraw Hill; Remington; and The Science and Practice of Pharmacy. Nineteenth Ed. Ed. Alfonso R. Gennaro Mack Publishing Co. Easton Pa.

### Additional Embodiments

Reference will now be made to more specific embodiments of the present disclosure and experimental results that provide support for such embodiments. However, Applicants note that the disclosure below is for illustrative purposes and is not intended to limit the scope of the claimed subject matter in any way.

### EXAMPLE 1

### Materials.

Reagents used in this study were purchased from Sigma Aldrich (St. Louis, Mo.) and were used without further purification: calcium chloride dihydrate (ACS Reagent, 99+%), sodium oxalate (Na₂C₂O, >99%), oxalic acid (99%), malonic acid (99%), succinic acid (99%), glutaric acid (99%), adipic acid (99%), tricarballylic acid (99%), butanetetracarboxylic acid (99%), DL-malic acid (99%), potassium tartarate dibasic hemihydrate (99%), dimethyl hydroxyglutarate (98%), DL-isocitric acid trisodium salt hydrate (93%), sodium citrate dehydrate (99%), potassium hydroxycitrate tribasic monohydrate (95%). Sodium chloride (99.9% ultrapure bioreagent) was purchased from JT Baker.

### EXAMPLE 2

### Calcium Oxalate Monohydrate (COM) Bulk Crystallization.

Crystallization was carried out in a 20-mL glass vial by dissolving NaCl in deionized water then adding 0.7 mL of 10 mM CaCl₂ stock solution which was prepared in advance. The sample vial was place in an oven set to 60° C. for one hour to ensure the crystallization was performed at 60° C. Subsequently 0.7 mL of 10 mL Na₂C₂O₄ stock solution was added to the solution drop wise while the solution was stirred at 400 rpm. To investigate the effect of organic growth modifiers (OGMs) on COM crystallization, different concentrations of OGMs were added to the solution dropwise. Also, pH correcting agent i.e., NaOH was added at the end of crystallization process to regulate the pH of the solution in the presence of OGMs. The concentration of NaOH added was varied according to the concentration of OGMs used (See Table 1). A clean glass slide (ca. 1.3×1.3 cm²) was placed at the bottom of the glass vial to readily collect the crystals prior to placing the vial in the oven. Final solution composition was set to be 0.7 mM CaCl₂:0.7 mM Na₂C₂O₄:15 mM NaCl and the amount of deionized water was adjusted to set the final volume to 10 mL. Crystallization was performed at 60° C. for three days without agitation. The glass slide was removed from the solution and dried at room temperature prior to analysis.

### EXAMPLE 3

### Calcium Oxalate Monohydrate (COM) Crystal Morphology Observation.

Morphology of COM crystals prepared in the absence and in the presence of OGMs were assessed and compared under optical microscope (Leica DM2500-M). Images were obtained in brightfield using reflectance mode and the images of crystals were measured the length (L, [001]), width (W, [010]) and aspect ratio (AR, [001]/[010]).

### EXAMPLE 4

### Calcium Ion Selective Electrode (ISE) Measurement.

A calcium ion selective electrode (ISE, ThermoScientific with Orion 9720BNWP ionplus® electrode) was used to measure the effect of OGMs on the kinetics of COM crystallization which measures the concentration of free calcium ions in solution. Samples were prepared in the similar manner as COM bulk crystallization. Final solution composition was set to be 0.5 mM CaCl₂:0.5 mM Na₂C₂O₄:150 mM NaCl and the measurement was performed at room temperature. During ISE measurements, the stir rate was adjusted to 1200 rpm to minimize the induction time for nucleation. OGM efficacy was measured by calculating the temporal depletion of calcium ion concentration (ppm range). The data was normalized by subtracting the concentration of the initial time point from each time points. The rate of depletion was calculated by measuring the initial slope of the crystal growth curve. The efficacy of OGMs was assessed using the percent inhibition, which was calculated by comparing the reduced slopes for each OGM relative to that of the control (i.e. absence of OGM). Prior to ISE measurements, the electrode was calibrated using a standard calcium solution (0.1 M, Orion Ion Plus), which was diluted with deionized water to three concentrations: 10⁻⁴, 10⁻³ and 10⁻² M. The ionic strength of each solution was adjusted using a standard solution (ISA, Thermo Scientific), which was added in a 1:50 volume ratio of ISA-to-standard.

### EXAMPLE 5

### Atomic Force Microscopy (AFM).

Atomic force microscopy (AFM, Bruker Multimode 4) was used to study the COM crystal surface topography at a nanometer length scale. Samples were mounted on a disk covered with a thin layer of thermally curable epoxy (MasterBond EP21AOLV). The epoxy was partially cured in an oven for ca. 20 min at 60° C. prior to transferring the crystals obtained from bulk crystallization. The crystals were immobilized on the epoxy with the [100] or [010] direction oriented normal to the specimen surface by placing the sample in an oven at 60° C. for two to three hours to completely cure the epoxy. The cantilevers used in this study were silicon nitride probes with gold reflex coating and a spring constant of 0.15 N/m (Olympus, TR800PSA). AFM was also employed to observe the kinetics of crystal growth in supersaturated calcium oxalate solutions by monitoring the velocity of step advancement on the (010) surface of COM crystals in real time. The experiment was designed to assess the effects of selected OGMs on step growth using a fluid cell (Bruker, MTFML) to produce an environment that mimics in situ crystallization. The fluid cell had two ports for inlet and outlet flow to maintain constant supersaturation during AFM measurements. A silicon O-ring was used to ensure no liquid leakage would occur while solutions were being continuously replenished. A dual syringe pump (CHEMYX, Fusion 200) was used to flow the solutions into the fluid cell.

### EXAMPLE 6

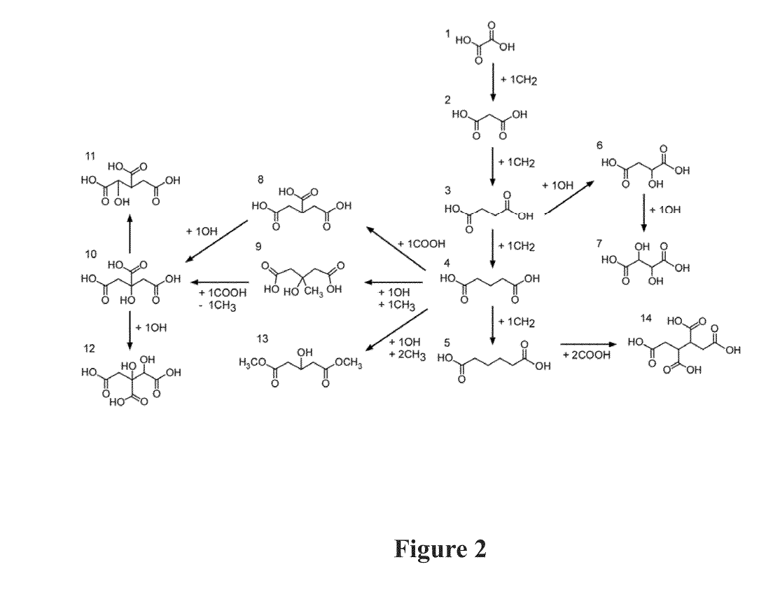

In attempt to systematically investigate the effect of functional groups in growth modifiers of calcium oxalate monohydrate (COM) crystals, a series of commercially available small organics were selected. The effect of molecules in terms of 1) increasing the number of carbons in the hydrocarbon backbone chain, 2) increasing the number of hydroxyl groups, and 3) increasing number of carboxyl groups (FIG. 2), were observed. Citrate, which is the main ingredient in the over-the-counter supplement for kidney stone patients, is known to have moderate inhibitory effect on COM crystallization. Citrate was utilized as a benchmark to evaluate the degree of inhibitory effect of the molecules.

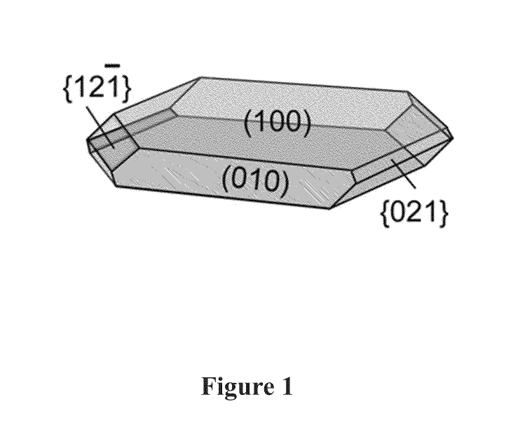

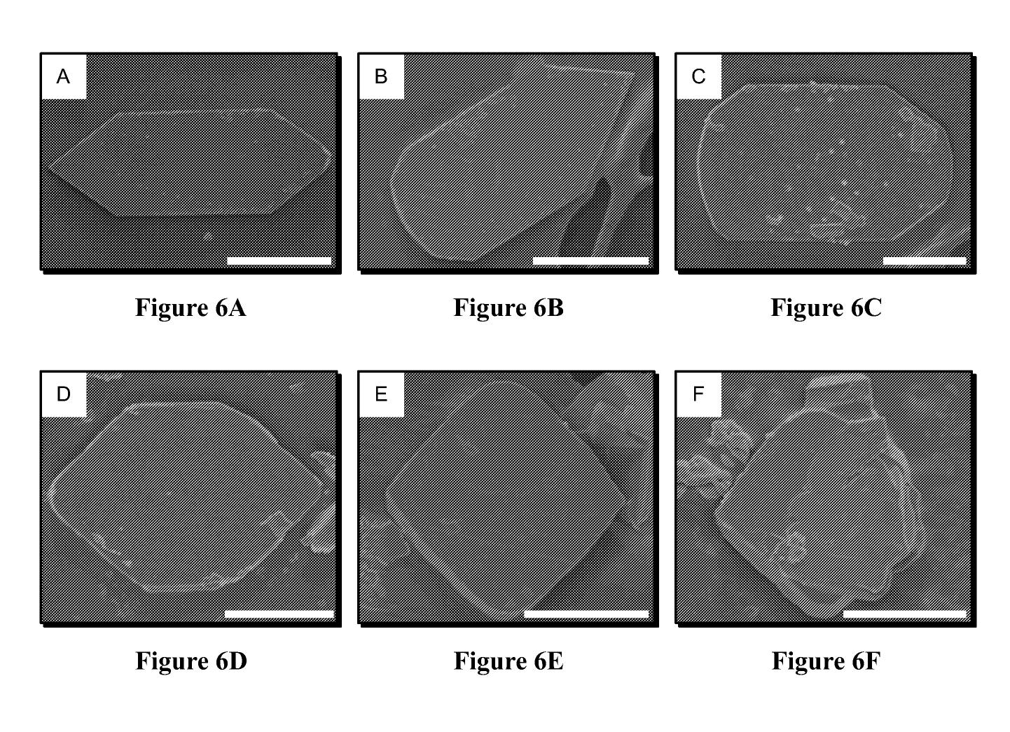

The crystallization method adopted results in COM crystals exhibiting hexagonal platelets with large (100) basal surface (FIGS. 1 and 6A). In the presence of inhibitors, the aspect ratio crystals may increase or decrease depending on the binding specificity of the molecules to the crystal surface. For instance, in the presence of 60 μg/mL HCA, the resulting COM crystals exhibited diamond shapes which indicates the preferential binding of HCA to {12-1} and/or {021} faces of COM crystals.

### EXAMPLE 7

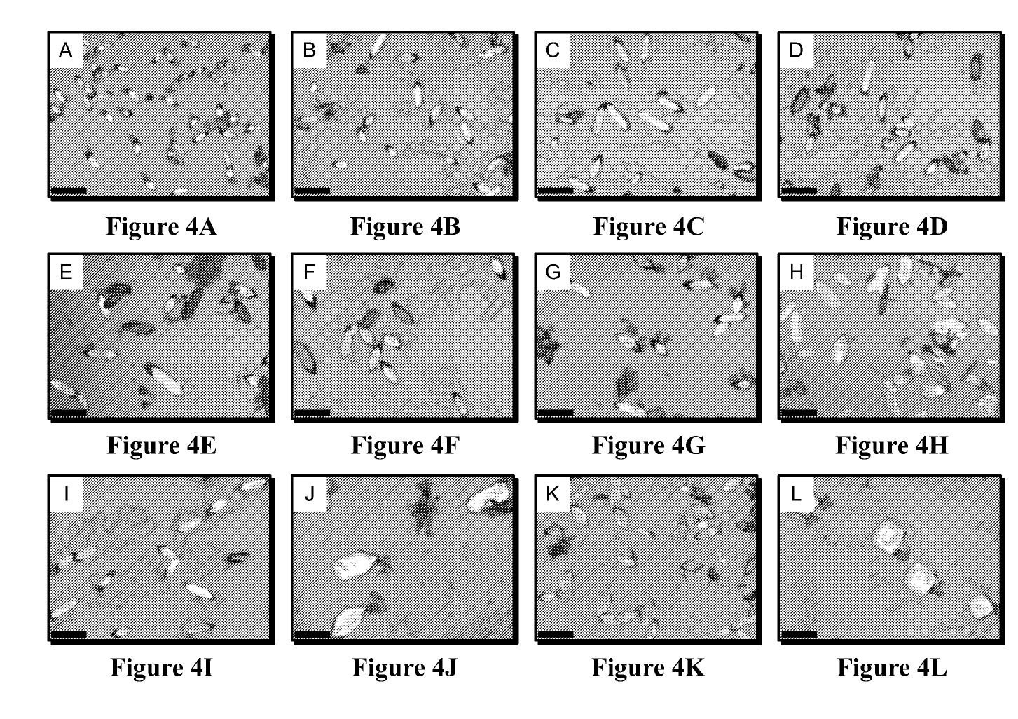

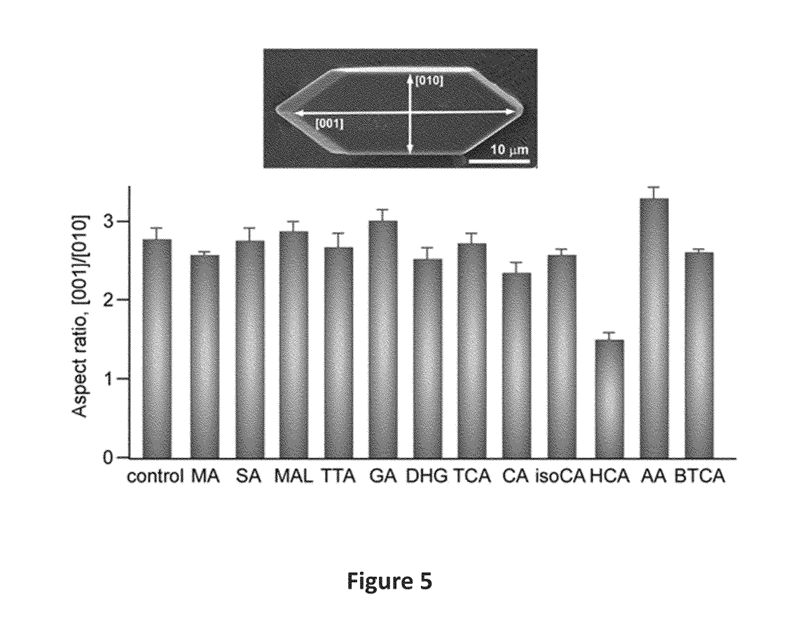

The morphology of the resulting COM crystals formed in the presence of small organics (60 μg/mL) was compared in terms of aspect ratio (FIGS. 4, 5) and several small organics were selected to observe the potency. COM crystallization in the presence of citrate (CA) and hydroxycitrate (HCA) at different concentrations (20, 60, 100 μg/mL) were observed (FIG. 6). COM crystals in the presence of increasing concentration of CA resulted in gradual decrease of the aspect ratio and rounding of apical tips. In the presence of increasing concentration of HCA, the aspect ratio of COM crystals was greatly decreased which indicates strong binding interaction of HCA to {12-1} and/or {021} COM crystal faces.

### EXAMPLE 8

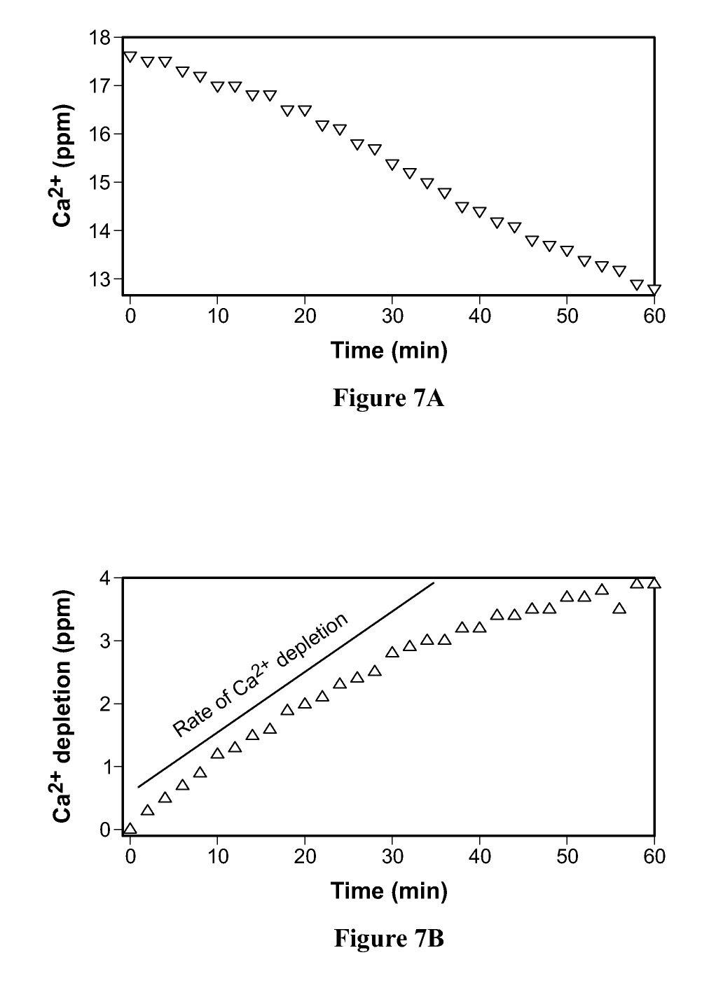

Additionally, the inhibitory efficacy of these small organics in COM crystallization was observed. An ion selective electrode measures the free calcium ion in the solution in the course of experiments. Efficacy of the small organics was measured by calculating the temporal depletion of calcium ion concentration. The data was normalized by subtracting the concentration of the initial time point from each time points. The rate of depletion was calculated by measuring the initial slope of the crystal growth curve. The efficacy of the small organics molecules was assessed using the percent inhibition, which was calculated by comparing the reduced slopes for each molecule relative to that of the control (FIG. 7).

### EXAMPLE 9

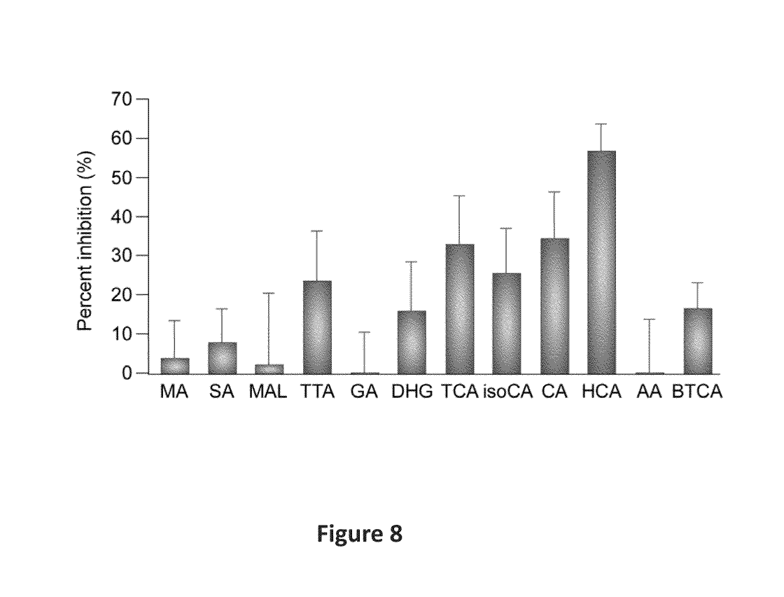

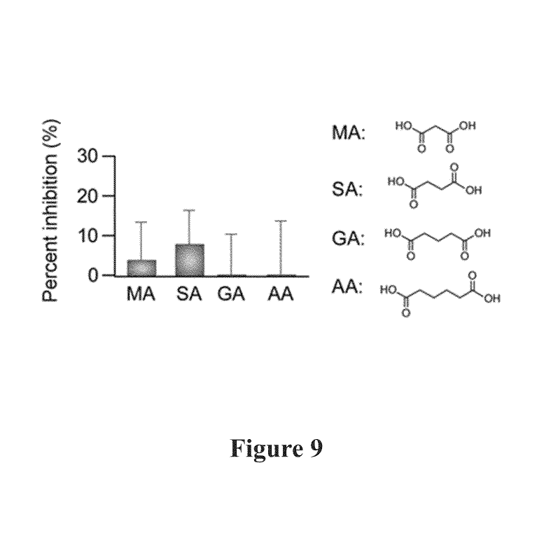

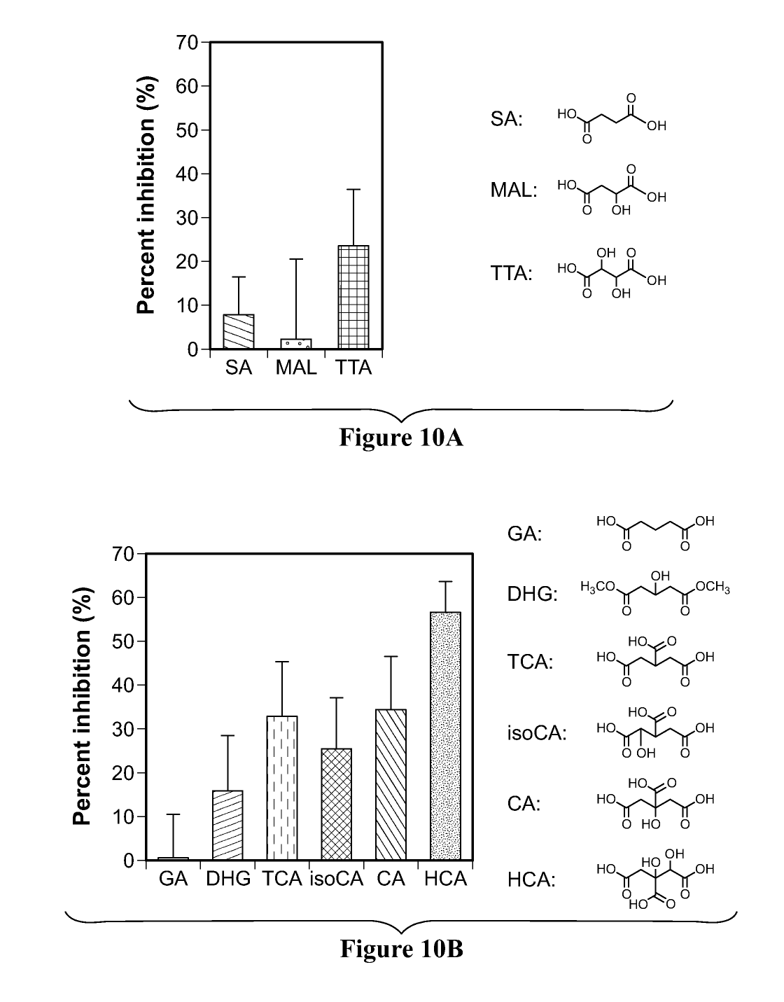

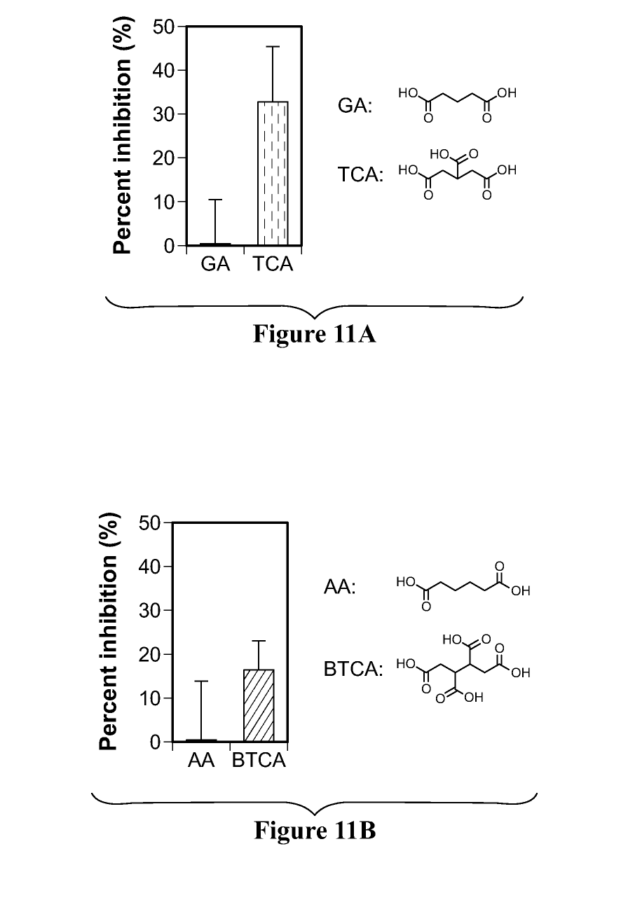

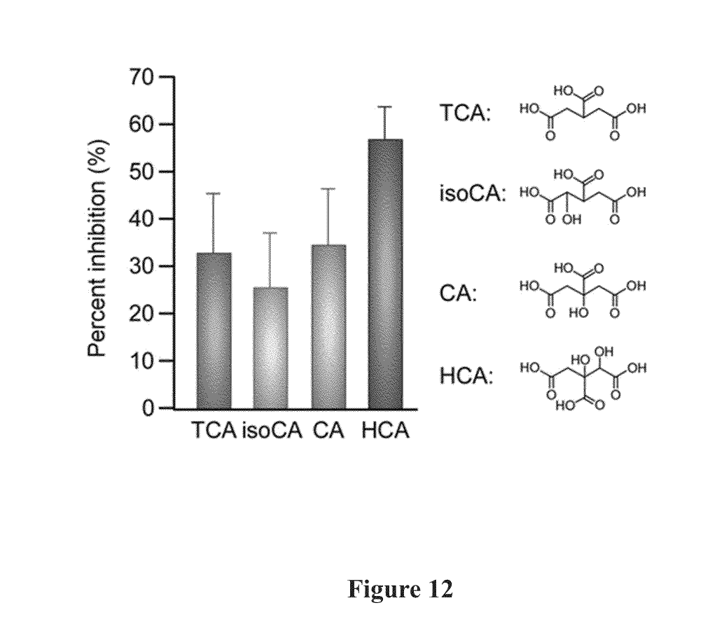

The efficacy of each molecule in inhibition of COM crystallization was compared in terms of the length of the carbon backbone chain, the number of hydroxyl group, and the number of carboxyl group (FIGS. 8-12). With regard to the number of carbons in the backbone assessed in this study, changes from (C₃ to C₆) showed little effect on the efficacy (FIG. 9). With regard to the number of hydroxyl group, the results suggest that hydroxyl groups can enhance the efficacy, but are not the sole contributors of a molecule's ability to inhibit COM growth. Also the additional hydroxyl group may have caused steric hindrance resulting in lower efficacy (FIG. 10). In the case of the number of carboxyl group, the results suggest that additional carboxyl group enhances the efficacy of a molecule.

### EXAMPLE 10

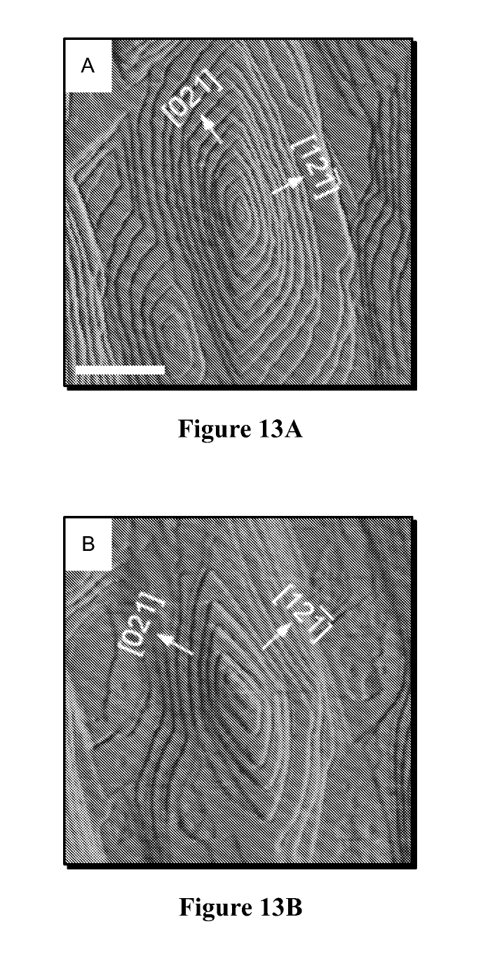

In order to investigate the interaction between the small organics and COM crystal surface at a molecular level, atomic force microscopy was utilized. The growth of hillocks by screw dislocation on the (010) face of COM crystals was measured in real time in the absence and in the presence of 0.1 μg/mL HCA (FIG. 13). Growth hillocks on (010) face are bounded by {021} and {12-1} faces. The shape of growth hillock in the presence of 0.1 μg/mL HCA suggests that HCA binds to {021} faces more strongly.

### EXAMPLE 11

### Citrate Congeners Upper Limit of Metastability

Four human urine samples, two from healthy normal subjects and two from patients with kidney stones, were obtained. Each urine sample was brought to pH 5.7. A 2 ml aliquot of the urine was put in a cuvette and stirred constantly with a magnetic stirrer. Absorbance at 620 nm was monitored continuously for increase in turbidity. Every 3 minutes a small aliquot of CaCl₂ solution was added to the wells. Three urine aliquots were run simultaneously (one control and two with the urine spiked with the anion of interest). Samples were run until a definitive change in absorbance was observed. Results are expressed as the change in nucleation induction time with inhibitor compared to the control without inhibitor. Each anion was added to the urine to increase concentration by 1 mM. It is to be noted that native urine will contain citrate and isocitrate. No hydroxycitrate is expected. Additionally, there are no reports, to date of tricarballylic acid measurements in human urine.

Table 1 shows results obtained from ANOVA comparing tricarballylic acid, hydroxycitrate, isocitrate, and citrate:

| Anion | Delay in nucleation (sec) | P value (compared to HCA) |
|---|---|---|
| Tricarbalyllate | 64 ± 70 | 0.03 |
| Isocitrate | 25 ± 66 | <0.01 |
| Citrate | 171 ± 70 | 0.27 |
| Hydroxycitrate | 350 ± 66 | NA |

Data is least squared means from ANOVA. Mean ± SEM.
P value for overall ANOVA is <0.01
Pairwise comparisons for the 4 anions:

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A composition for inhibiting calcium oxalate crystal growth comprising:
at least one citrate derivative of an organic acid; and
at least one pharmaceutically acceptable carrier.

2. The composition of claim 1, wherein the organic acid that is a citrate derivative is hydroxycitrate.

3. The composition of claim 1, wherein the citrate derivative of an organic acid is tricarballylic acid.

4. The composition of claim 1, wherein the citrate derivative of an organic acid is isocitrate.

5. The composition of claim 1, comprising two citrate derivatives of an organic acid.

6. The composition of claim 5, wherein the two citrate derivatives of an organic acid are hydroxycitrate and tricarballylic acid.

7. The composition of claim 5, wherein the two citrate derivatives of an organic acid are hydroxycitrate and isocitrate.

8. The composition of claim 1, further comprising a urinary protein.

9. The composition of claim 1, wherein the composition is in a form suitable for oral administration.

10. The composition of claim 1, wherein the composition inhibits growth of the crystals by about 60%.

11. The composition of claim 1, wherein the composition inhibits growth of crystals comprising calcium oxalate monohydrate.

12. The composition of claim 1, wherein the composition inhibits growth of crystals comprising calcium oxalate dihydrate.

13. The composition of claim 1, wherein the composition inhibits growth of crystals comprising calcium oxalate trihydrate.

14. The composition of claim 1, wherein the composition inhibits growth of crystals comprising calcium oxalate monohydrate and calcium oxalate dihydrate.

15. A composition comprising:
at least one citrate derivative of an organic acid; and
at least one pharmaceutically acceptable carrier, wherein the composition inhibits growth of crystals comprising calcium phosphate.

16. A method of controlling calcium oxalate crystal growth in a subject in need thereof comprising administering to the subject a composition comprising:
at least one citrate derivative of an organic acid; and
at least one pharmaceutically acceptable carrier.

17. The method of claim 16, wherein the subject has kidney stone disorder.

18. The method of claim 16, wherein the subject has renal calcification disorder.

19. The method of claim 16, wherein the subject has biomineralization induced disease.

20. The method of claim 16, further comprising administering the composition post lithotripsy.

21. The method of claim 16, wherein the organic acid that is a citrate derivative is hydroxycitrate.

22. The method of claim 16, wherein the citrate derivative of an organic acid is tricarballylic acid.

23. The method of claim 16, wherein the citrate derivative of an organic acid is isocitrate.

24. The method of claim 16, wherein the composition comprises two citrate derivatives of an organic acid.

25. The method of claim 24, wherein the two citrate derivatives of an organic acid are hydroxycitrate and tricarballylic acid.

26. The method of claim 24, wherein the two citrate derivatives of an organic acid are hydroxycitrate and isocitrate.

27. The method of claim 16, wherein the composition further comprises a urinary protein.

28. A method of treating kidney stone disorder comprising administering to a subject in need thereof a therapeutically effective amount of a composition comprising:
at least one citrate derivative of an organic acid; and
at least one pharmaceutically acceptable carrier.

29. The method of claim 28, wherein the composition further comprises a urinary protein.

30. A method of treating calcium oxalate stone disease comprising administering to a subject in need thereof a therapeutically effective amount of a composition comprising:
at least one citrate derivative of an organic acid; and
at least one pharmaceutically acceptable carrier.

31. The method of claim 30, wherein the composition comprises two citrate derivatives of an organic acid.
